(19) United States
(12) Patent Application Publication
(10) Pub. No.: US 2018/0125795 A1
(43) Pub. Date: May 10, 2018

(54) CINNAMALDEHYDE COMPOSITIONS AND METHODS

(71) Applicant: ThermoLife International, LLC, Phoenix, AZ (US)

(72) Inventors: Ronald Kramer, Phoeix, AZ (US); Alexandros Nikolaidis, Nea Kallikratia (GR)

(73) Assignee: ThermoLife International, LLC, Phoenix, AZ (US)

(21) Appl. No.: 15/861,567

(22) Filed: Jan. 3, 2018

Related U.S. Application Data
(60) Provisional application No. 62/441,879, filed on Jan. 3, 2017.

(51) Int. Cl. A61K 31/11; A61K 47/12; A61K 47/14; A61K 47/10; A61K 47/44; A61P 3/10; A61P 3/04

(52) U.S. Cl. CPC A61K 31/11; A61K 47/12; A61K 47/14; A61P 3/04; A61K 47/44; A61P 3/10; A61K 47/10

(57) ABSTRACT

Disclosed are solid cinnamaldehyde compositions and methods for producing solid cinnamaldehyde compositions that are virtually flavorless.

### CROSS REFERENCE TO RELATED APPLICATIONS

This document claims priority to and the benefit of the filing date of U.S. Provisional Patent Application 62/441,879 titled “CINNAMALDEHYDE COMPOSITIONS AND METHODS” to Alexander Nikolaidis and Ronald Kramer, which was filed on Jan. 3, 2017, the disclosure of which is hereby incorporated entirely herein by reference.

### BACKGROUND

Cinnamaldehyde is the organic compound that gives cinnamon its flavor and odor. The compound is found in the bark of cinnamon trees and other species of the genus Cinnamomum as a pale yellow, viscous liquid. This essential oil of cinnamon bark contains about 98% cinnamaldehyde. A variety of processed foods also contain trace amounts of cinnamaldehyde, such as cinnamon rolls and orange juice (Friedman et al., “Cinnamaldehyde Content in Foods Determined by Gas Chromatography—Mass Spectrometry,” J Agric Food Chem., 2000, 48(11):5702-9). Friedman et al. attributed their observation to commercial cinnamon powder containing 8.2 to 27.5 mg cinnamaldehyde/g cinnamon powder. Cinnamaldehyde has also been found to be a potent antioxidant and antimicrobial in vitro (Singh et al., “A comparison of chemical, antioxidant and antimicrobial studies of cinnamon leaf and bark volatile oils, oleoresins and their constituents,” Food Chem Toxicol., 2007, 45(9):1650-61). In laboratory animals, cinnamaldehyde was found to have antidiabetic effects (Babu et al., “Cinnamaldehyde—a potential antidiabetic agent,” Phytomedicine, 2007, 14(1):15-22). However, cinnamaldehyde's effectiveness for lowering blood sugar and combating diabetes in humans has not been demonstrated. To date, it remains doubtful whether cinnamaldehyde would have any antidiabetic and glucose-lowering effects in humans. Cinnamaldehyde content in cinnamon preparations very low (typical commercial cinnamon products sold for glucose support contain 600-1200 mg of cinnamon, equaling 4.92-33 mg of cinnamaldehyde). Applicants found that the current top-selling cinnamon aquatic extract, which has been shown to be effective in humans (Ziegenfuss et al., “Effects of a Water-Soluble Cinnamon Extract on Body Composition and Features of the Metabolic Syndrome in Pre-Diabetic Men and Women,” J Int Soc Sports Nutr., 2006, 3:45-53) and is used in many nutritional supplements to promote post exercise recovery, creatine absorption, and blood glucose control, contains no cinnamaldehyde.

In part because of the uncertain effectiveness in humans, pure cinnamaldehyde has never been utilized in the production of medicine or supplements for use by humans, for example, people who need to reduce their blood glucose and Hemoglobin A1c levels, for example, diabetics; who want to prevent diabetes, have a better weight, have a better glucose metabolism, increase muscle mass gain; or who want antioxidant protection or any other of the myriad possible health benefits of cinnamaldehyde.

Another deterrent for the use of cinnamaldehyde by humans is the pharmacotechnical problems and side effects associated with its use. Cinnamaldehyde is a liquid at room temperature with a melting point of between −4° C. and −9° C. The liquid state is very impractical for use in the various solid pharmaceutical forms most medicine and supplements are manufactured, such as tablets, powders, capsules and the like. The liquid state is also very problematic to handle during drug and supplement manufacture, because it is also a sticky volatile liquid. It is also difficult to make salt forms of cinnamaldehyde, because it is neither an acid nor a base. Thus, it is impossible to form salts with cinnamaldehyde, which is the usual way of transforming liquid active compounds into solid forms. Pure liquid cinnamaldehyde having a very pungent and spicy flavor (Fenaroli's Handbook of Flavor Ingredients. Volume 2. Edited, translated, and revised by T. E. Furia and N. Bellanca. 2nd ed. Cleveland: The Chemical Rubber Co., 1975, p. 91). Pure liquid cinnamaldehyde is unpleasant for humans to consume at the relatively large doses needed to achieve a pharmacological effect. Additionally, pure cinnamaldehyde irritates the epithelium, for example, of the airways and the gastrointestinal tract.

In view of these obstacles, there is a need for ways of administering cinnamaldehyde that reduce irritation to the gastrointestinal tract and the unpleasant taste and/or smell of cinnamaldehyde.

### SUMMARY

This disclosure relates to methods of producing solid compositions comprising cinnamaldehyde or its derivatives and a carrier. The carrier may be selected from the group consisting of: a high-molecular weight fatty acid, hydrogenated derivative of the high-molecular weight fatty acid, esterified derivative of the high-molecular weight fatty acid, an aliphatic alcohol, hydrogenated derivative of the aliphatic alcohol, esterified derivative of the aliphatic alcohol, resin, wax, and mixtures thereof.

In one aspect, the method comprises the steps of: heating a carrier until it reaches its melting temperature point to produce a melted carrier; mixing liquid cinnamaldehyde into the melted carrier to produce a mixture; cooling the mixture; milling the cooled mixture to a desirable particle size; and washing off under filtration the mixture with a solvent until the washed off solute lacks a pungent smell of cinnamon, wherein the solvent is miscible with cinnamaldehyde.

In another aspect, the method consists of: dissolving a carrier in a first solvent; mixing the dissolved carrier and liquid cinnamaldehyde to produce a mixture; evaporating the first solvent from the mixture; milling the evaporated mixture in a desirable particle size; and washing off under filtration the mixture with second solvent until the washed off solute lacks the pungent smell of cinnamon, wherein the second solvent solubilizes cinnamaldehyde.

The first solvent can be evaporated under vacuum condition. In some implementations, the first solvent is selected from the group consisting of ethanol, hydroalcoholic mixtures, and water at alkaline pH. In some embodiments, the first solvent is ethanol. In some implementations, the second solvent is water at alkaline pH. In some aspects, the carrier is shellac and the solvent is water at alkaline pH. In some implementations, water is made alkaline with sodium bicarbonate.

This disclosure also relates to a solid composition comprising cinnamaldehyde or its derivatives. In one embodiment, the composition comprises cinnamaldehyde or its derivatives and a carrier, for example, the solid compositions produced according to the methods described above and in the Detailed Description. In one aspect, cinnamaldehyde is dispersed inside the composition which allows the composition to be less pungent and spicy in taste than liquid cinnamaldehyde, as well as cause less gastrointestinal disturbances than liquid cinnamaldehyde. Thus, the solid composition is safe for human consumption. In another aspect, the composition comprises cinnamaldehyde at an amount of 0.01%-50% by weight of the composition, for example, 2% or 20% by weight of the composition. In still another aspect, the composition is solid at temperatures of above −6° C., for example at room temperature. In some aspects, the solid cinnamaldehyde compositions are in powder form, which could be used in the production of nutraceuticals and capsules.

In some embodiments, the composition comprises at least 100 mg cinnamaldehyde and is formulated as solid single administration oral composition. In some aspects, such compositions further comprise a carbohydrate and/or a protein. In other aspects, such compositions further comprise creatine.

The carrier may be selected from the group consisting of: a high-molecular weight fatty acid, hydrogenated derivative of the high-molecular weight fatty acid, esterified derivative of the high-molecular weight fatty acid, an aliphatic alcohol, hydrogenated derivative of the aliphatic alcohol, esterified derivative of the aliphatic alcohol, resin, wax, and mixtures thereof. In some embodiments, the carrier may be a high-molecular weight fatty acid, such as stearic acid. In other embodiments, the carrier may be palmitic acid. In some embodiments, the carrier may be a mixture, for example, a mixture of steric acid and carnauba wax. In one implementation, the carrier is a mixture comprising 80% by weight stearic acid and 20% by weight carnauba wax.

In another embodiment, the invention is directed to methods of reducing blood glucose in a human subject; treating obesity and/or reducing fat mass in a human subject; or promoting post-exercise recovery. The methods comprise administering to the human subject a pharmaceutically effective amount of solid cinnamaldehyde. In some implementations, the pharmaceutically effective amount of solid cinnamaldehyde is at least 100 mg per day or at least 200 mg per day. In some preferred implementations, the solid cinnamaldehyde is prepared according to the methods of the invention. Notably, such administration of cinnamaldehyde causes no gastrointestinal side effects.

For methods of reducing blood glucose in a human subject, the human subject may be diabetic and cinnamaldehyde is preferably administered before a meal. In such implementations, cinnamaldehyde administration reduces the level of Hemoglobin A1c in the diabetic human subject. For methods of promoting post-exercise recovery, cinnamaldehyde is preferably administered within two hours, for example within one hour, after exercise. In some implementations, cinnamaldehyde is administered with a carbohydrate and/or a protein.

### DETAILED DESCRIPTION

This disclosure is directed to methods for producing solid cinnamaldehyde compositions safe for human consumption, for example, being suitable for use in solid supplement, medical, and food preparations. The compositions of cinnamaldehyde are solid at temperatures of above −6° C., for example, at room temperature.

The formulations of the present disclosure allow the inclusion of large doses of cinnamaldehyde, which was previously unachievable in the industry. Furthermore, these compositions contain a much higher percentage of cinnamaldehyde compared to solid sources containing liquid cinnamaldehyde in the prior art. The compositions are also very suitable and practical for formulation of drugs, supplements and foodstuffs containing cinnamaldehyde. In some embodiments, the amount of cinnamaldehyde in the composition is between 0.01% and 50% by weight of the composition. Specifically, cinnamaldehyde may be 2%, 5%, 10%, 15%, 20%, 25%, 30%, 35%, 40%, 45%, or 50% by weight of the composition. In some aspects, the solid cinnamaldehyde compositions are in powder form, which could be used in the production of nutraceuticals and capsules.

More importantly, the cinnamaldehyde compositions do not cause any deleterious side effects associated with cinnamaldehyde or cinnamon ingestion, such as a burning sensation and gastrointestinal disturbances. Notably, the formulations containing the cinnamaldehyde do not have an overwhelming cinnamon flavor, which would be typical of any regular formulation containing such high amounts of cinnamaldehyde. Finally, the formulations of the present disclosure are much more stable and contain the initial amount of cinnamaldehyde for much longer as compared to a solid form that contains liquid cinnamaldehyde.

The methods for producing solid cinnamaldehyde compositions generally comprise mixing cinnamaldehyde and a carrier in a liquid environment. The liquid environment may be provided by melting the carrier. Alternatively, the liquid environment may be provided to dissolving the carrier in a solvent (for example a first solvent) that is miscible with cinnamaldehyde. Examples of such a solvent include ethanol, hydroalcoholic mixtures, and water at alkaline pH.

As used herein, the term “water” refer to unaltered water, which has a pH of around 7 and up to 7.4. As used herein, “water at alkaline pH” refers to water with pH that is higher than 7.4. Water at alkaline pH can be made by mixing water with a suitable base, for example, sodium bicarbonate, calcium hydroxide, or magnesium hydroxide.

After the carrier and cinnamaldehyde are mixed in the liquid environment, the mixture is cooled or dried to a solid then milled to the desired particle size. After which, the milled solid is washed with a solvent (in some implementations, the second solvent) that is miscible with cinnamaldehyde but does not solubilize the carrier (for example, water). The washing step is performed with a filter to separate the milled solid from the washed off solute. The washing step may also be performed under vacuum conditions. The washing step is performed until the washed off solute lacks the pungent smell of cinnamon. At this point, all of the unincorporated cinnamaldehyde was been washed away to leave only incorporated cinnamaldehyde in the milled solid. In some implementations, the washed off solute is collected and dried to collect the unincorporated cinnamaldehyde.

The compositions of the invention comprises cinnamaldehyde and a carrier, such as a carrier that is suitable for human consumption. In some embodiments, the ratio of cinnamaldehyde to the carrier is 4:1.

The carrier is solid at room temperature and melts in temperatures not exceeding 295° C. In some embodiments, the carrier has a melting point below 248° C., has no offensive taste, is solid at temperatures up to 45° C., is practically insoluble in water/saliva, and has no chemical incompatibilities with cinnamaldehyde. In some implementations, the carrier is soluble in evaporable solvents. An exemplary carrier is a high-molecular weight fatty acid, such as, tridecylic acid (C13), myristic acid (C14), pentadecanoic acid (C15), palmitic acid (C16), margaric acid (C17), stearic acid (C18), nonadecylic acid (C19), arachidic acid (C20), heneicosylic acid (C21), behenic acid (C22), tricosylic acid (C23), lignoceric acid (C24), pentacosylic acid (C25), cerotic acid (C26), heptacosylic acid (C27), montanic acid (C28), nonacosylic acid (C29), melissic acid (C30), hentriacontylic acid (C31), lacceroic acid (C32), psyllid acid (C33), geddic acid (C34), ceroplastic acid (C35), hexatriacontylic acid (C36), heptatriacontanoic acid (C37), and octatriacontanoic acid (C38). The carrier may also be a hydrogenated monounsaturated and polyunsaturated high-molecular weight fatty acid, such as hydrogenated soybean oil. High-molecular weight alcohols, such as stearyl alcohol and lauryl alcohol, may be a carrier. Esters of aforementioned alcohols and acids, such as lauryl stearate, may also be a carrier. Waxes, such as bee wax and carnauba wax, and resins, such as shellac and mastic gum, can also be used as a carrier. The carrier may also be a composition of the aforementioned examples. Therefore, the carrier may be selected from the group consisting of: a high-molecular weight fatty acid, hydrogenated derivative of the high-molecular weight fatty acid, esterified derivative of the high-molecular weight fatty acid, an aliphatic alcohol, hydrogenated derivative of the aliphatic alcohol, esterified derivative of the aliphatic alcohol, resin, wax, and mixtures thereof. For example, the carrier may be a composition comprising a high-molecular weight fatty acid and a wax, such as stearic acid and carnauba wax. In one embodiment, the carrier may a mixture of 80% by weight stearic acid and 20% by weight carnauba wax.

In some embodiments, the composition comprises additives that can enhance pharmaceutical or organoleptic properties, (e.g. one of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and combinations thereof) and/or a carrier (e.g. one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof). These additives may be solids or liquids, and the type of additive may be generally chosen based on the type of administration being used. Those of ordinary skill in the art will be able to readily select suitable pharmaceutically effective additives from the disclosure in this document. In particular implementations, pharmaceutically acceptable additives may include, by non-limiting example, calcium phosphate, cellulose, stearic acid, croscarmelose cellulose, magnesium stearate, and silicon dioxide.

In some aspects, the composition further comprises at least one other compound that helps with blood glucose control and diabetes management. The other compound may be a part of the solid cinnamaldehyde matrix or in a formulation with the solid cinnamaldehyde. The other compound can include: agaricus mushroom, alpha lipoic acid, American ginseng, astragalus, berberine, alcohol, blond psyllium, caffeine, chromium in all its forms, coffee, fenugreek, flaxseed, glucomannan, guarumo, ivy gourd, magnesium, milk thistle, niacin and niacinamide, oats, prickly pear cactus, soy and soy products (such as soy protein), white mulberry, xantham gum, beta carotene, black tea, conjugated linoleic acid, cranberry, omega-3 fats, garlic, jambolan, lycopene, selenium, aloe, apple cider vinegar, artemisia herba alba, ashwaghanda, banaba, bay leaf, bean pod, beta glucans, bilberry, biotin, bitter melon, bitter orange, black psyllum blueberry, branch chained amino acids, burdock, calcium, cinnamon and its extracts and constituents other than cinnamaldehyde (such as cinnamic acid), chanca piedra, chia, cocoa, coenzyme Q10, creatine, diacylglycerol, fig leaf extracts, folic acid, ginger, panax ginseng, green tea, guar gum, hesperidin, holy basil, Irvingia gabonensis, jackfruit, kudzu, L-arginine, L-carnitine, lutein, lysine, maitake mushroom, myrcia, olive oil, oolong tea, papaya, polydextrose, pycnogenol, red yeast, reishi mushroom, resveratrol, safflower, salacia, sesame and its byproducts, stevia, stinging nettle, taurine, turmeric, vanadium, vitamin C, vitamin D, vitamin E, vitamin K, white horehound, yerba mate, or inorganic nitrates such as potassium nitrate or arginine nitrate.

The invention is also directed to methods of using cinnamaldehyde-containing compositions to control blood sugar levels and reduce high blood sugar-related complications; to enhance glucose transportation in the muscle and creatine muscle delivery, endurance, athletic performance, and strength; to improve body composition; and to promote post-exercise recovery. In some implementations, the methods comprise administering cinnamaldehyde to promote muscle anabolism, exercise recovery, and reduce delayed onset muscle soreness (DOMS), for example when cinnamaldehyde is administered after exercise.

With regard to methods of improving body composition, the administration of cinnamaldehyde increases the percentage of muscle and decrease the amount of fat in the human subject in need of improved body composition, for example a human subject whose body mass index indicates the subject is overweight (BMI >25).

The administration of cinnamaldehyde promotes post-exercise recovery by reducing the time for a subject's oxygen consumption level (VO₂) to return to his or her pre-exercise level when cinnamaldehyde is administered after exercise. In these implementations, cinnamaldehyde is administered with a protein and/or a carbohydrate. The protein may be any protein commonly used in nutritional supplement manufacture, which includes but is not limited to whey protein, milk protein, casein protein, beef protein, soy protein, hemp protein, rice protein, canola protein, spirulina, and pea protein. The carbohydrate may by any commonly used form of simple or complex carbohydrates used in the manufacture of dietary supplements, which includes but is not limited glucose, fructose, dextrose, palatinose, maltodextrin, various starches, activated barley, waxy maize, cereals, and oats. In a preferred embodiment, cinnamaldehyde is administered to the human subject within two hours of completion of exercise, for example within 90 minutes, 60 minutes, or 30 minutes of completion of exercise.

In particular, the capsule product helps these people control blood sugar levels and reduce high blood sugar related complications.

In another implementation, cinnamaldehyde is administered with creatine, preferably with a carbohydrate, to improve the absorption of creatine by the muscle. As used herein, the term “creatine” refers to any nutritionally acceptable form of creatine which includes but is not limited to anhydrous creatine, creatine monohydrate, creatine nitrate, creatine hydrochloride, creatine citrate, creatine pyruvate, kre-alkalyn buffered creatine, creatine sulfate, creatine malate, creatine ethyl ester hydrochloride, creatine ethyl ester malate, creatine phosphate, creatine gluconate, polyethylene glycosylated creatine, cyclocreatine, creatinol-o-phosphate, or a creatine amides with an amino acid.

### Examples

The present disclosure is further illustrated by the following examples that should not be construed as limiting. The contents of all references, patents, and published patent applications cited throughout this application, as well as the Figures, are incorporated herein by reference in their entirety for all purposes.

I. a Sample Preparation Method Using Stearic Acid as the Carrier:

1. Measure 80 grams of stearic acid and 20 grams of liquid cinnamaldehyde.

2. Melt stearic acid at 69.3° C.

3. Slowly pour the liquid cinnamaldehyde into the melted stearic acid. Mix continuously for at least 5 minutes.

4. Cool down the mixture of liquid cinnamaldehyde and stearic acid.

5. Mill the mixture into fine powder using a pharmaceutical mill.

6. Put the powder in a filter funnel and wash off under vacuum with water for 5 minutes.

7. Air dry the powder.

Stearic acid is safe to use in foods, relatively inexpensive, stable in storage, and practically insoluble in water. It has a melting point of 69.3° C., which is not so hot as to destroy many temperature-sensitive ingredients.

II. A Sample Preparation Method Using Shellac as the Carrier:

1. Dissolve 50 grams shellac and 20 grams of cinnamaldehyde in 250 ml of ethanol.

2. Mix until both the shellac and the cinnamaldehyde are totally dissolved.

3. Evaporate the ethanol at 60° C. under vacuum until a solid is formed.

4. Mill the solid into powder.

5. Wash the powder on a paper filter with water for at least 5 minutes.

III. Components of a Powder Formulation Containing Solid Cinnamaldehyde (Amounts Per Serving):

1. Creatine Nitrate 3 grams

2. 20% cinnamaldehyde in Stearic Acid carrier 500 mg

3. Taurine 1 gram

4. Dextrose 20 grams

The powder formulation is used for enhancing glucose transportation in the muscle and creatine muscle delivery, as well as endurance, athletic performance, strength and improving body composition.

IV. Components of Capsule Product Formulation (Amounts Per Serving):

1. 20% cinnamaldehyde in Stearic Acid carrier—1000 mg

2. Chromium Picolinate—100 mcg

3. Vanadium Sulfate—500 mcg

4. Banaba Leaf Extract—500 mg

5. Propionyl-L-Carnitine Nitrate—1000 mg

The capsule product is useful for people suffering from diabetes. In particular, the capsule product helps these people control blood sugar levels and reduce high blood sugar related complications.

V. Components of a Cinnamaldehyde-Containing Composition for Promoting Post-Exercise Recovery:

1. Cinnamaldehyde 20% in stearic acid—500 mg

2. Whey Protein—30,000 mg

3. Glucose—10,000 mg

VI. Effects of Cinnamaldehyde on Blood Glucose Level and Glucose Tolerance:

In one human subject, ingestion of one gram of a 20% cinnamaldehyde in stearic acid composition in the morning resulted in a decrease of 24 mg/dl blood glucose from 86 mg/dl (fasting blood glucose level). The subject reported the need to consume carbohydrates to avoid dizziness. In a glucose tolerance test, the ingestion of one gram of the composition after overnight fasting and ingestion of 100 g glucose dissolved in 500 ml water resulted in a 20% drop in blood glucose level 90 minutes after ingestion of the sugar water (132 mg/dl versus 108 mg/dl).

VII. Effects of Cinnamaldehyde on Post-Exercise Recovery:

Administration of cinnamaldehyde to a human subject after the human subject completed an exercise session reduced the time for the human subject's whole-body oxygen consumption to be return to pre-exercise levels. The effect of cinnamaldehyde on promoting post-exercise exercise is best when cinnamaldehyde is administered within an hour after exercise and its effect is reduced when administered two hours after exercise. The effect of cinnamaldehyde on promoting post-exercise exercise is enhanced when it is administered with a protein and/or a carbohydrate (for example, a combination of whey powder and glucose).

1. A method of producing solid cinnamaldehyde, the method comprising:
heating a carrier until it reaches its melting temperature point to produce a melted carrier;
mixing liquid cinnamaldehyde into the melted carrier to produce a mixture;
cooling the mixture;
milling the cooled mixture to a desirable particle size; and
washing off under filtration the mixture with a solvent until the washed off solute lacks a pungent smell of cinnamon, wherein the solvent is miscible with cinnamaldehyde.

2. A method of producing solid cinnamaldehyde, the method consisting of:
dissolving a carrier in a first solvent;
mixing the dissolved carrier and liquid cinnamaldehyde to produce a mixture;
evaporating the first solvent from the mixture;
milling the evaporated mixture in a desirable particle size; and
washing off under filtration the mixture with second solvent until the washed off solute lacks the pungent smell of cinnamon, wherein the second solvent solubilizes cinnamaldehyde.

3. The method of claim 2, wherein the first solvent is selected from the group consisting of:
ethanol, hydroalcoholic mixtures, and water at alkaline pH.

4. The method of claim 3, wherein the water at alkaline pH is made alkaline with sodium bicarbonate.

5. The method of claim 2, wherein the first solvent is evaporated under vacuum condition.

6. The method of claim 2, wherein the first solvent is ethanol.

7. The method of claim 1 wherein the solvent is water.

8. The method of claim 2, wherein the second solvent is water.

9. The method of claim 2, wherein the carrier is shellac, the first solvent is water at alkaline pH, and the second solvent is water.

10. A solid composition produced according to the method of claim 1.

11. A solid composition produced according to the method of claim 2.

12. A solid composition comprising at least 2% by weight cinnamaldehyde, wherein the solid composition does not any gastrointestinal side effects upon ingestion.

13. The composition of claim 12, wherein the composition is solid in temperatures of at or above −6° C.

14. The composition of claim 13, wherein the composition is solid in temperatures of at room temperature.

15. The composition of claim 12, wherein the composition is safe for human consumption.

16. The composition of claim 12, wherein the composition is a powder suitable for the production of nutraceuticals and capsules.

17. A solid composition comprising:
cinnamaldehyde or one or more of its derivatives; and
a carrier, wherein the carrier is a solid at room temperature,
wherein cinnamaldehyde is dispersed inside the composition and the composition is less pungent and spicy in taste than liquid cinnamaldehyde.

18. The composition of claim 16, wherein the composition causes less gastrointestinal disturbances than liquid cinnamaldehyde.

19. The composition of claim 16, wherein the carrier is selected from the group consisting of: high-molecular weight fatty acid, aliphatic alcohol, hydrogenated and esterified derivative of a high-molecular weight fatty acid, hydrogenated and esterified derivative of an aliphatic alcohol, resin, wax, and mixtures thereof.

20. The composition of claim 18, wherein the high-molecular weight fatty acid is stearic acid.

21. The composition of claim 19, wherein the cinnamaldehyde is between 0.01% and 50% by weight of the composition.

22. The composition of claim 20, wherein the cinnamaldehyde 20% by weight of the composition.

23. The composition of claim 16, wherein the carrier is a mixture comprising 80% by weight stearic acid and 20% by weight carnauba wax.

24. The composition of claim 16, wherein the carrier is palmitic acid.

25. The composition of claim 16, wherein the composition is a powder suitable for the production of nutraceuticals and capsules.

26. A method of reducing blood glucose in a human subject, the method comprising administering orally a pharmaceutically effective amount of solid cinnamaldehyde.

27. The method of claim 26, wherein the pharmaceutically effective amount of solid cinnamaldehyde is at least 100 mg per day, wherein the administration of cinnamaldehyde causes no gastrointestinal side effects.

28. The method of claim 27, wherein the pharmaceutically effective amount of solid cinnamaldehyde is at least 200 mg per day, wherein the administration of cinnamaldehyde causes no gastrointestinal side effects.

29. The method of claim 26, wherein solid cinnamaldehyde is produced according to the method of claim 1.

30. The method of claim 26, wherein the solid cinnamaldehyde is produced according to the method of claim 2.

31. The method of claim 26, wherein the human subject is diabetic.

32. The method of claim 31, wherein cinnamaldehyde administration reduces the level of Hemoglobin A1c in the human subject.

33. The method of claim 31, wherein the human subject is administered solid cinnamaldehyde before a meal.

34. A method of treating obesity and/or reducing fat mass in a human subject in need thereof, the method comprising administering to the human subject a pharmaceutically effective amount of solid cinnamaldehyde.

35. The method of claim 34, wherein the pharmaceutically effective amount of solid cinnamaldehyde is at least 200 mg/day.

36. The method of claim 34, wherein the solid cinnamaldehyde is produced according to the method of claim 1.

37. The method of claim 34, wherein the solid cinnamaldehyde is produced according to the method of claim 2.

38. The method of claim 34, wherein the administration of cinnamaldehyde causes no gastrointestinal side effects.

39. A method of promoting post exercise recovery, the method comprising administering to a subject a solid oral cinnamaldehyde composition within two hours after exercise.

40. The method of claim 39, wherein the cinnamaldehyde amount given is at least 100 mg

41. The method of claim 39, further comprising administering a carbohydrate and/or a protein, wherein the cinnamaldehyde is administered with the carbohydrate and/or the protein.

42. The composition of claim 17, wherein the composition comprises at least 100 mg cinnamaldehyde, the composition further comprises a carbohydrate and/or a protein and is formulated as solid single administration oral composition.

43. The composition of claim 17, wherein the composition comprises at least 100 mg cinnamaldehyde, the composition further comprises creatine and is formulated as a solid single administration oral composition.
